(19) United States
(12) Patent Application Publication
(10) Pub. No.: US 2007/0270952 A1
(43) Pub. Date: Nov. 22, 2007

(54) PROSTHETIC INTERVERTEBRAL DISCS IMPLANTABLE BY MINIMALLY INVASIVE SURGICAL TECHNIQUES

(75) Inventors: Elizabeth V. Wistrom, Palo Alto, CA (US); Darin C. Gittings, Sunnyvale, CA (US); Jeffrey J. Dolin, Belmont, CA (US); Michael L. Reo, Redwood City, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: Spinal Kinetics, Inc.

(21) Appl. No.: 11/379,340

(22) Filed: Apr. 19, 2006

(51) Int. Cl. A61F 2/44

(52) U.S. Cl. 623/17.11

(57) ABSTRACT

Prosthetic intervertebral discs and methods for using the same are described. The subject prosthetic discs include upper and lower endplates separated by a compressible core member. The subject prosthetic discs exhibit stiffness in the vertical direction, torsional stiffness, bending stiffness in the saggital plane, and bending stiffness in the front plane, where the degree of these features can be controlled independently by adjusting the components of the discs. The subject prosthetic discs have shapes, sizes, and other features that make them particularly suitable for deployment using minimally invasive surgical procedures.

### BACKGROUND OF THE INVENTION

The intervertebral disc is an anatomically and functionally complex joint. The intervertebral disc is composed of three component structures: (1) the nucleus pulposus; (2) the annulus fibrosus; and (3) the vertebral endplates. The biomedical composition and anatomical arrangements within these component structures are related to the biomechanical function of the disc.

The spinal disc may be displaced or damaged due to trauma or a disease process. If displacement or damage occurs, the nucleus pulposus may herniate and protrude into the vertebral canal or intervertebral foramen. Such deformation is known as herniated or slipped disc. A herniated or slipped disc may press upon the spinal nerve that exits the vertebral canal through the partially obstructed foramen, causing pain or paralysis in the area of its distribution.

To alleviate this condition, it may be necessary to remove the involved disc surgically and fuse the two adjacent vertebra. In this procedure, a spacer is inserted in the place originally occupied by the disc and it is secured between the neighboring vertebrae by the screws and plates/rods attached to the vertebra. Despite the excellent short-term results of such a “spinal fusion” for traumatic and degenerative spinal disorders, long-term studies have shown that alteration of the biomechanical environment leads to degenerative changes at adjacent mobile segments. The adjacent discs have increased motion and stress due to the increased stiffness of the fused segment. In the long term, this change in the mechanics of the motion of the spine causes these adjacent discs to degenerate.

To circumvent this problem, an artificial intervertebral disc replacement has been proposed as an alternative approach to spinal fusion. Although various types of artificial intervertebral discs have been developed to restore the normal kinematics and load-sharing properties of the natural intervertebral disc, they can be grouped into two categories, i.e., ball and socket joint type discs and elastic rubber type discs.

Artificial discs of ball and socket type are usually composed of metal plates, one to be attached to the upper vertebra and the other to be attached to the lower vertebra, and a polyethylene core working as a ball. The metal plates have concave areas to house the polyethylene core. The ball and socket type allows free rotation between the vertebrae between which the disc is installed. Artificial discs of this type have a very high stiffness in the vertical direction; they cannot replicate the normal compressive stiffness of the natural disc. Also, the lack of load bearing capability in these types of discs causes adjacent discs to take up the extra loads resulting in the eventual degeneration of the adjacent discs.

In elastic rubber type artificial discs, an elastomeric polymer is embedded between metal plates and these metal plates are fixed to the upper and the lower vertebrae. The elastomeric polymer is bonded to the metal plates by having the interface surface of the metal plates be rough and porous. This type of disc can absorb a shock in the vertical direction and has a load bearing capability. However, this structure has a problem in the interface between the elastomeric polymer and the metal plates. Even though the interface surfaces of the metal plates are treated for better bonding, polymeric debris may nonetheless be generated after long term usage. Furthermore, the elastomer tends to rupture after a long usage because of its insufficient shear-fatigue strength.

Because of the above described disadvantages associated with either the ball/socket or elastic rubber type discs, there is a continued need for the development of new prosthetic devices.

### SUMMARY OF THE INVENTION

Prosthetic intervertebral discs and methods for using such discs are provided. The subject prosthetic discs include an upper endplate, a lower endplate, and a compressible core member disposed between the two endplates. The prosthetic discs are provided having shapes, sizes, and other features that are particularly suited for implantation using minimally invasive surgical procedures known to those skilled in the art.

In a first aspect, the subject prosthetic discs are characterized by including top and bottom endplates separated by one or more compressible core members. The two plates are held together by at least one fiber wound around at least one region of the top endplate and at least one region of the bottom endplate. The subject discs may include integrated vertebral body fixation elements. When considering a lumber disc replacement from the posterior access, the two plates are preferably elongated, having a length that is substantially greater than its width, Typically the dimensions of the prosthetic discs will range in height from 8 mm to 15 mm, while the width can range from 6 mm to 13 mm. Preferably the height of the prosthetic discs will range from 9 mm to 11 mm while the preferable widths are 10 mm to 12 mm. The length of the prosthetic discs can range from 18 mm to 30 mm, with the preferable size being 24 mm to 28 mm. Typical shapes include oblong, bullet-shaped, lozenge-shaped, rectangular, or the like

In several embodiments, the disc structures preferably are held together by at least one fiber wound around at least one region of the upper endplate and at least one region of the lower endplate. The fibers are generally high tenacity fibers with a high modulus of elasticity. The elastic properties of the fibers, as well as factors such as the number of fibers used, the thickness of the fibers, the number of layers of fiber windings, the tension applied to each layer, and the crossing pattern of the fiber windings enable the prosthetic disc structure to mimic the functional characteristics and biomechanics of a normal-functioning, natural disc.

A conventional approach can be used to place the pair of prosthetic discs, including the posterior lumbar interbody fusion (PLIF) and transforaminal lumbar interbody fusion (TLIF) procedures. Apparatus and methods for implanting prosthetic intervertebral discs using minimally invasive surgical procedures are also provided. In one embodiment, the apparatus includes a pair of cannulas that are inserted posteriorly, side-by-side, to gain access to the spinal column at the disc space. A pair of prosthetic discs are implanted by way of the cannulas to be located between two vertebral bodies in the spinal column. In another embodiment, a single, selectively expandable disc is employed. In an unexpanded state, the disc has a relatively small profile to facilitate delivery of it to the disc space. Once operatively positioned, it can then be selectively expanded to an appropriate size to adequately occupy the disc space. Implantation of the single disc involves use of a single cannula and an articulating chisel or a chisel otherwise configured to establish a curved or right angle disc delivery path so that the disc is substantially centrally positioned in the disc space. Preferably, the prosthetic discs have sizes and structures particularly adapted for implantation by the minimally invasive procedure.

Other and additional devices, apparatus, structures, and methods are described by reference to the drawings and detailed descriptions below.

### BRIEF DESCRIPTIONS OF THE FIGURES

The Figures contained herein are not necessarily drawn to scale, with some components and features being exaggerated for clarity.

FIG. 1 provides an illustration of a minimally invasive surgical procedure for implanting a pair of prosthetic discs.

FIG. 2 provides an illustration of an alternative minimally invasive surgical procedure for implanting a prosthetic disc.

FIG. 3A provides a three-dimensional view (in partial cross-section) of a preferred prosthetic disc for use with a minimally invasive surgical procedure.

FIG. 3B provides a three-dimensional view (in partial cross-section) of another preferred prosthetic disc for use with a minimally invasive surgical procedure.

FIGS. 4A-E illustrate another preferred prosthetic disc and several of its component parts.

FIGS. 5A-B illustrate another preferred prosthetic disc and one endplate thereof.

FIGS. 6A-C illustrate another preferred prosthetic disc and one endplate thereof.

FIGS. 7-10 illustrate several alternative endplate structures for incorporation into a full prosthetic disc such as those illustrated in FIGS. 3A-B.

FIGS. 11A-D illustrate another preferred prosthetic disc and two endplates thereof.

### DESCRIPTIONS OF THE PREFERRED EMBODIMENTS

Prosthetic intervertebral discs, methods of using such discs, apparatus for implanting such discs, and methods for implanting such discs are described herein. It is to be understood that the prosthetic intervertebral discs, implantation apparatus, and methods are not limited to the particular embodiments described, as these may, of course, vary. It is also to be understood that the terminology used herein is for the purpose of describing particular embodiments only, and is not intended to be limiting, since the scope of the present inventions will be limited only by the appended claims.

Insertion of the prosthetic discs may be approached using a conventional procedure, such as a posterior lumbar interbody fusion (PLIF) or transforaminal lumbar interbody fusion (TLIF). For the PLIF procedure the spine is approached via midline incision in the back and the erector spinae muscles are stripped bilaterally from the vertebral lamina at the required levels. A laminectomy is then performed to allow visualization of the nerve roots. A partial facetectomy may also be performed to facilitate exposure. The nerve roots are retracted to one side and a discectomy is performed. Optionally, a chisel may then used to create a groove(s) in the vertebral endplates to accept the fixation means of the prosthesis(es). An appropriately-sized prosthesis(es) is then inserted into the intervertebral space on either side of the vertebral canal.

The TLIF procedure is also a posterior approach, but differs from the PLIF procedure in that the entire facet joint is removed and the access is only on one side of the vertebral body. After the facetectomy, the discectomy is performed. Optionally, a chisel may then used to create a groove(s) in the vertebral endplates to accept the fixation means of the prosthesis(es). The prosthesis(es) is then inserted into the intervertebral space. One prosthesis may be moved to the contralateral side of the access and then a second prosthesis can be inserted on the access side.

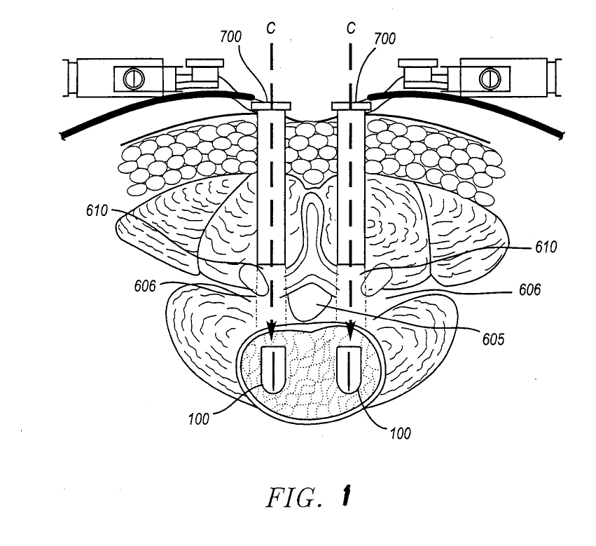

Turning to the Figures, a minimally invasive surgical procedure for implanting a pair of intervertebral discs is illustrated in FIG. 1. The minimally invasive surgical implantation method may be performed using a posterior approach, rather than the anterior approach used for conventional lumbar disc replacement surgery or the PLIF and TLIF procedures described above. Turning to FIG. 1, a pair of cannulae 700 is inserted posteriorly to provide access to the spinal column. More particularly, a small incision is made and a pair of access windows is created through the lamina 610 of one of the vertebrae on each side of the vertebral canal to access the natural vertebral disc to be replaced. The spinal cord 605 and nerve roots 606 are avoided or mobilized to provide access. Once access is obtained, each of the cannulae 700 is inserted. The cannulae 700 may be used to remove the natural disc by conventional means. Alternatively, the natural disc may have already been removed by other means prior to insertion of the cannulae.

Once the natural disc has been removed and the cannulae 700 located in place, a pair of prosthetic discs are implanted between adjacent vertebral bodies. In the preferred embodiment, the prosthetic discs have a shape and size adapted for the minimally invasive procedure, such as the elongated one-piece prosthetic discs described hereinbelow. A prosthetic disc 100 is guided through each of the two cannulas 700 (see arrows “C” in FIG. 1) such that each of the prosthetic discs is implanted between the two adjacent vertebral bodies. In the preferred method, the two prosthetic discs 100 are located side by side and spaced slightly apart between the two vertebrae. Optionally, prior to implantation, grooves are created on the internal surfaces of one or both of the vertebral bodies in order to engage anchoring features located on the prosthetic discs 100. The grooves may be created using a chisel tool adapted for use with the minimally invasive procedure, i.e., adapted to extend through a relatively small access space and to provide the chisel function within the intervertebral space after removal of the natural disc.

Optionally, a third prosthetic disc may be implanted using the methods described above. The third prosthetic disc is preferably implanted at a center point, between the two prosthetic discs 100 shown in FIG. 1. The third disc would be implanted prior to the two discs shown in the Figure. Preferably, the disc would be implanted by way of either one of the cannulas, then rotated by 90° to its final load bearing position between the other two prosthetic discs. The first two prosthetic discs 100 would then be implanted using the method described above.

Additional prosthetic discs may also be implanted in order to obtain desired performance characteristics, and the implanted discs may be implanted having many different relative orientations within the intervertebral space. In addition, the multiple prosthetic discs may each have different performance characteristics. For example, a prosthetic disc to be implanted in the central portion of the intervertebral space may be more resistant to compression than one or more prosthetic discs that are implanted more near the outer edge of the intervertebral space. This resistance to compression can be in the range where the discs that are implanted more near the outer edge of the intervertebral space are approximately 80% the stiffness of the central portion to approximately 5% the stiffness of the central portion, preferably in the range of 60% to 30%. Other performance characteristics may be varied as well.

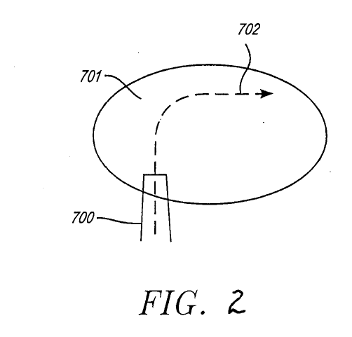

An alternative minimally invasive implantation method and apparatus is illustrated schematically in FIG. 2. In this alternative implantation method, a single cannula 700 is used. The cannula is inserted on one side of the vertebral canal in the manner described above. Once the cannula is inserted, a chisel may be used to create a groove 701 having a 90° bend on the endplates of the two adjacent vertebral bodies. Thus, the terminal portion of the groove 702 is perpendicular to the axis defined by the insertion cannula 700.

As summarized above, the subject invention is also directed to prosthetic intervertebral discs. By prosthetic intervertebral disc is meant an artificial or manmade device that is configured or shaped so that it can be employed as a total or partial replacement for an intervertebral disc in the spine of a vertebrate organism, e.g., a mammal, such as a human. The subject prosthetic intervertebral discs have dimensions that permit them, either alone or in combination with one or more other prosthetic discs, to substantially occupy the space between two adjacent vertebral bodies that is present when the naturally occurring disc between the two adjacent bodies is removed, i.e., a void disc space. By substantially occupy is meant that it occupies at least about 50% by surface area, such as at least about 80% by surface area or more. The subject discs may have a roughly bullet or lozenge shaped structure adapted to facilitate implantation by minimally invasive surgical procedures.

The subject discs are characterized in that they include both an upper (or top) and lower (or bottom) endplate, where the upper and lower endplates are separated from each other by a compressible element such as one or more core members, where the combination structure of the endplates and compressible element provides a prosthetic disc that functionally closely mimics a natural disc. A preferred feature of the preferred subject prosthetic discs is that the top and bottom endplates are preferably held together by at least one fiber wound around at least one portion of each of the top and bottom endplates. As such, the two endplates (or planar substrates) are held to each other by one or more fibers that are wrapped around at least one domain/portion/area of the upper endplate and lower endplate such that the plates are joined to each other.

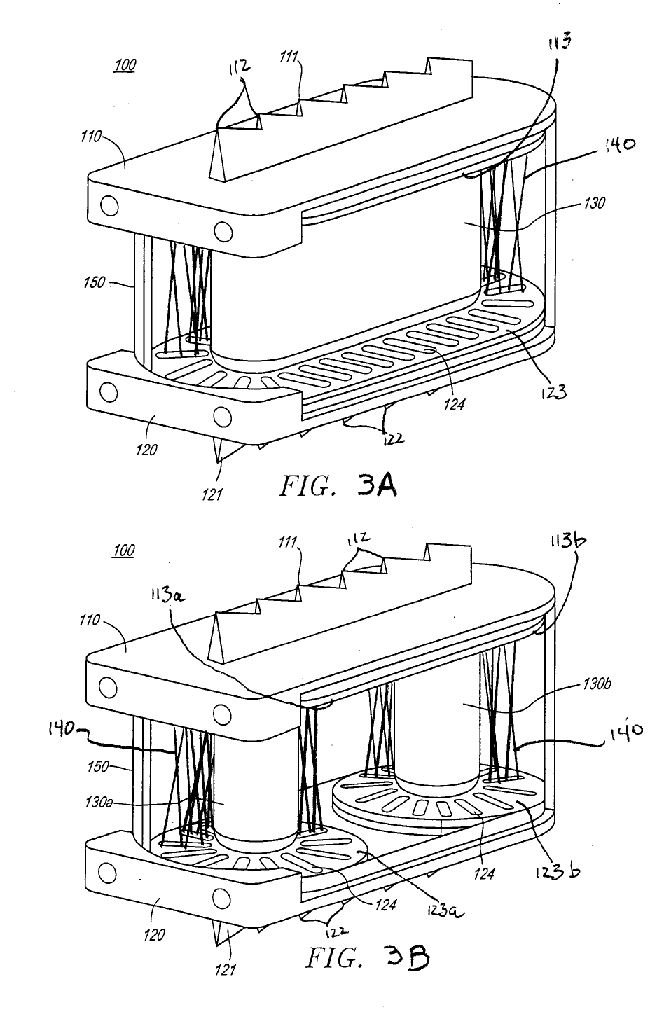

Two different representative intervertebral discs are shown in FIGS. 3A and 3B. As can be seen, the prosthetic discs 100 each include a top endplate 110 and a lower endplate 120. A core member 130 (FIG. 3A) or a pair of core members 13a-b (FIG. 3B) is located between the top endplate 110 and lower endplate 120. The top and bottom endplates 110 and 120 are typically generally planar substrates having a length of from about 12 mm to about 45 mm, such as from about 13 mm to about 44 mm, a width of from about 11 mm to about 28 mm, such as from about 12 mm to about 25 mm, and a thickness of from about 0.5 mm to about 5 mm, such as from about 1 mm to about 3 mm. The top and bottom endplates are fabricated from a physiologically acceptable material that provides for the requisite mechanical properties, primarily structural rigidity and durability. Representative materials from which the endplates may be fabricated are known to those of skill in the art and include, but are not limited to: metals such as titanium, titanium alloys, stainless steel, cobalt/chromium, etc.; plastics such as polyethylene with ultra high molar mass (molecular weight) (UHMW-PE), polyether ether ketone (PEEK), etc.; ceramics; graphite; etc.

The discs also preferably include fibers 140 wound between and connecting the upper endplate 110 to the lower endplate 120. Preferably, the fibers 140 extend through a plurality of apertures 124 formed on portions of each of the upper and lower endplates 110, 120. Thus, a fiber 140 extends between the pair of endplates 110, 120, and extends up through a first aperture 124 in the upper endplate 110 and back down through an adjacent aperture 124 in the upper endplate 110. (For clarity, the fibers 140 are not shown extending all the way around the cores 130, 130a-b in FIGS. 3A-B. Nor are the fibers 140 shown in all of the Figures. Nevertheless, fibers 140, as shown, for example, in FIGS. 3A-B, are present in and perform similar functions in each of the embodiments described below.) The fibers 140 preferably are not tightly wound, thereby allowing a degree of axial rotation, bending, flexion, and extension by and between the endplates. The amount of axial rotation generally has a range from about 0° to about 15°, preferably from about 2° to 10°. The amount of bending generally has a range from about 0° to about 18°, preferably from about 2° to 15°. The amount of flexion and extension generally has a range from about 0° to about 25°, preferably from about 3° to 15°. The core members 130, 130a-b, may be provided in an uncompressed or a pre-compressed state. An annular capsule 150 is optionally provided in the space between the upper and lower endplates, surrounding the core member(s) 130, 130a-b, and the fibers 140.

In the example shown in FIG. 3A, a single elongated core member 130 is provided, whereas the example structure shown in FIG. 3B has a dual core including two generally cylindrical core members 130a, 130b. It is believed that the dual core structure (FIG. 3B) better simulates the performance characteristics of a natural disc. In addition, the dual core structure is believed to provide less stress on the fibers 140 relative to the single core structure (FIG. 3A). Each of the exemplary prosthetic discs shown in FIGS. 3A-B has a greater length than width. Exemplary shapes to provide these relative dimensions include rectangular, oval, bullet-shaped, lozenge-shaped, or others. This shape facilitates implantation of the discs by the minimally invasive procedures described above in relation to FIG. 1.

The upper surface of the upper endplate 110 and the lower surface of the lower endplate 120 are preferably each provided with a fixation mechanism for securing the endplate to the respective opposed surfaces of the upper and lower vertebral bodies between which the prosthetic disc is to be installed. For example, in FIG. 3A-B, the upper endplate 110 includes an anchoring feature 111. The anchoring feature 111 is intended to engage a mating groove that is formed on the surface of the vertebral body to thereby secure the endplate to its respective vertebral body. The anchoring feature 111 extends generally perpendicularly from the generally planar external surface of the upper endplate 110, i.e., upward from the upper side of the endplate as shown in FIGS. 3A-B. The anchoring feature 111 has a plurality of serrations 112 located on its top edge. The serrations 112 are intended to enhance the ability of the anchoring feature to engage the vertebral body and to thereby secure the upper endplate 110 to the spine.

Similarly, the lower surface of the lower endplate 120 includes an anchoring feature(s) 121. The anchoring feature(s) 121 on the lower surface of the lower endplate 120 may be identical in structure and function to the anchoring feature(s) 111 on the upper surface of the upper endplate 110, including or with the exception of its location on the prosthetic disc. The anchoring feature(s)121 on the lower endplate 120 is intended to engage a mating groove formed on the lower vertebral body, whereas the anchoring feature(s)111 on the upper endplate 110 is intended to engage a mating groove on the upper vertebral body. Thus, the prosthetic disc 100 is held in place between the adjacent vertebral bodies.

The anchoring feature(s) 111, 121 may optionally be provided with one or more aspects such as holes, slots, ridges, grooves, indentations or raised surface(s) (not shown). The aspects will anchor the prosthetic disc 100 to the vertebral bodies by allowing for bony ingrowth. In addition, more anchoring features may be provided on either or both of the upper and lower endplates 110, 120. Each endplate 110, 120 may have a different number of anchoring features, and the anchoring features may have a different orientation on each endplate. The number of anchoring features generally ranges in number from about 0 to about 500, preferably from about 1 to 10. Alternatively, another fixation mechanism may be used, such as ridges, knurled surfaces, serrations, or the like. In still other embodiments, no external fixation mechanism is used, and the disc(s) are held in place laterally by the friction forces imparted to the disc by the vertebral bodies.

As noted above, the upper endplate 110 and lower endplate 120 each contain a plurality of apertures 124 through which the fibers 140 may be passed through or wound, as shown. The actual number of apertures 124 contained on the endplate is variable. Increasing the number of apertures allows an increase in the circumferential density of the fibers holding the endplates together. The number of apertures generally ranges from about 3 to 100 apertures, preferably in the range of 10 to 30. In addition, the shape of the apertures may be selected so as to provide a variable width along the length of the aperture. For example, the width of the apertures may taper from a wider inner end to a narrow outer end, or visa versa. Additionally, the fibers may be wound multiple times within the same aperture, thereby increasing the radial density of the fibers. In each case, this improves the wear resistance and increases the torsional and flexural stiffness of the prosthetic disc, thereby further approximating natural disc stiffness. In addition, the fibers 140 may be passed through or wound on each aperture, or only on selected apertures, as needed. The fibers may be wound in a uni-directional manner, where the fibers are wound in the same direction, e.g., clockwise, which closely mimics natural annular fibers found in a natural disc, or the fibers may be wound bi-directionally. Other winding patterns, either single or multi-directional, are also possible.

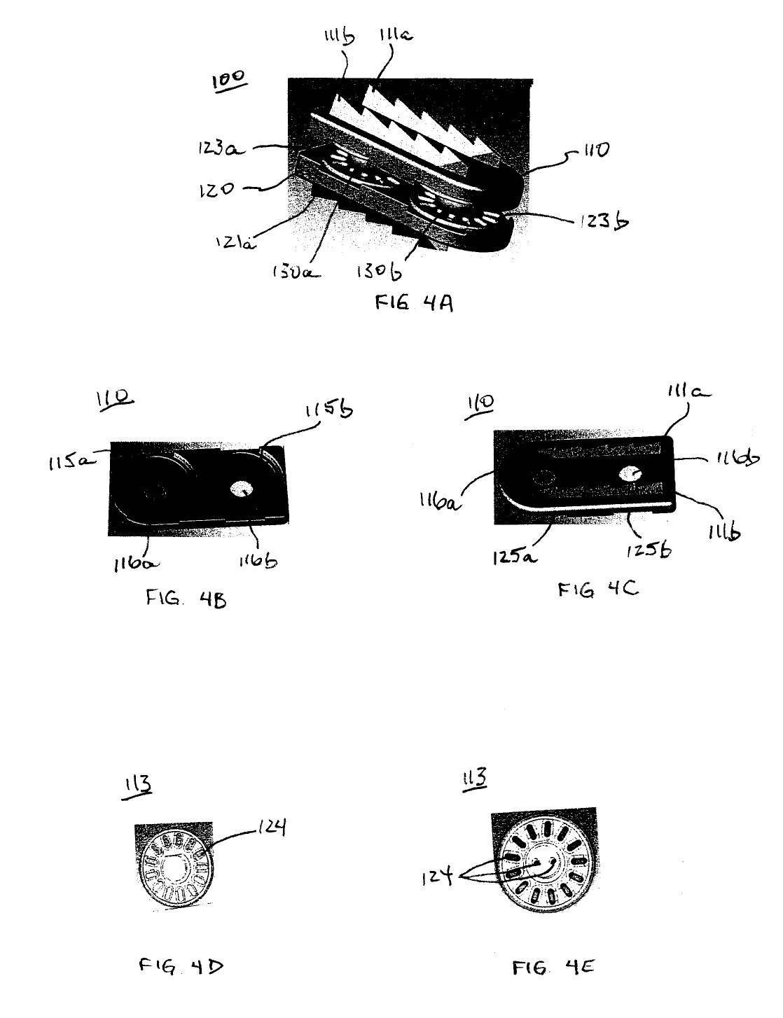

In several of the preferred embodiments, the apertures 124 are substantially displaced from the edges of the endplates. For example, in the embodiments illustrated in FIGS. 3B and 4A, many of the apertures 124 extend generally through the center of the endplates 110, 120, and are therefore substantially displaced from the edges thereof. Similarly, in the embodiments shown in FIGS. 5A-B, 7, 9, and 10, many of the apertures 124 are spaced substantially away from the longitudinal ends of each of the endplates 110, 120. This displacement of the apertures 124 from the edges of the endplates provides the prosthetic disc with a footprint that is based upon the shape and size of the endplates without requiring that the fiber winding be limited to placement on the edges of those endplates.

One purpose of the fibers 140 is to hold the upper endplate 110 and lower endplate 120 together and to limit the range-of-motion to mimic the range-of-motion of a natural disc. Accordingly, the fibers preferably comprise high tenacity fibers with a high modulus of elasticity, for example, at least about 100 MPa, and preferably at least about 500 MPa. By high tenacity fibers is meant fibers that can withstand a longitudinal stress of at least 50 MPa, and preferably at least 250 MPa, without tearing. The fibers 140 are generally elongate fibers having a diameter that ranges from about 100 μm to about 1000 μm, and preferably about 200 μm to about 400 μm. Optionally, the fibers may be injection molded with an elastomer to encapsulate the fibers, thereby providing protection from tissue ingrowth and improving torsional and flexural stiffness, or the fibers may be coated with one or more other materials to improve fiber stiffness and wear. Additionally, the core may be injected with a wetting agent such as saline to wet the fibers and facilitate the mimicking of the viscoelastic properties of a natural disc.

The fibers 140 may be fabricated from any suitable material. Examples of suitable materials include polyester (e.g., Dacron®), polyethylene (including, for example, ultra-high molecular weight polyethelene (UHMWPE)), polyaramid, poly-paraphenylene terephthalamide (e.g., Kevlar®), carbon or glass fibers, polyethylene terephthalate, acrylic polymers, methacrylic polymers, polyurethane, polyurea, polyolefin, halogenated polyolefin, polysaccharide, vinylic polymer, polyphosphazene, polysiloxane, and the like.

The fibers 140 may be terminated on an endplate by tying a knot in the fiber on the superior or inferior surface of an endplate. Alternatively, the fibers 140 may be terminated on an endplate by slipping the terminal end of the fiber into a aperture on an edge of an endplate, similar to the manner in which thread is retained on a thread spool. The aperture may hold the fiber with a crimp of the aperture structure itself, or by an additional retainer such as a ferrule crimp. As a further alternative, tab-like crimps may be machined into or welded onto the endplate structure to secure the terminal end of the fiber. The fiber may then be closed within the crimp to secure it. As a still further alternatives, a polymer may be used to secure the fiber to the endplate by welding, including adhesives or thermal bonding. The polymer would preferably be of the same material as the fiber (e.g., UHMWPE, PE, PET, or the other materials listed above). Still further, the fiber may be retained on the endplates by crimping a cross-member to the fiber creating a T-joint, or by crimping a ball to the fiber to create a ball joint.

In the embodiments shown in FIGS. 3A-B, each of the upper endplate 110 and lower endplate 120 is provided with one or more inner assemblies 113, 123, respectively. Each of the inner assemblies 113, 123 forms a portion of its respective endplate and is the structural member that includes the apertures 124 through which the fibers 140 are preferably wound. For example, in FIG. 3A, each inner assembly 113, 123 is generally oval in shape to fit generally within its respective endplate 110, 120. In FIG. 3B, on the other hand, each inner assembly 113a-b, 123a-b is generally round and occupies less than one-half of the length of the respective endplate 110, 120. Other shapes and sizes for the inner assemblies 113, 123 are possible. Preferably, each inner assembly 113, 123 is welded or otherwise structurally connected to its respective endplate 110, 120. The inner assemblies 113, 123 may be formed of any of the materials described above as being proper for use in constructing the endplates.

The core member(s) 130, 130a-b are intended to provide support to and to maintain the relative spacing between the upper endplate 110 and lower endplate 120. The core members 130, 130a-b are made of a relatively compliant material, for example, polyurethane or silicone, and are typically fabricated by injection molding. A preferred construction for the core member includes a nucleus formed of a hydrogel and an elastomer reinforced fiber annulus. For example, the nucleus, the central portion of the core member 130, may comprise a hydrogel material such as a water absorbing polyurethane, polyvinyl alcohol (PVA), polyethylene oxide (PEO), polyvinylpyrrolidone (PVP), polyacrylamide, silicone, or PEO based polyurethane. The annulus may comprise an elastomer, such as silicone, polyurethane or polyester (e.g., Hytrel®), reinforced with a fiber, such as polyethylene (e.g., ultra high molecular weight polyethylene, UHMWPE), polyethylene terephthalate, or poly-paraphenylene terephthalamide (e.g., Kevlar®).

The shape of each of the core members 130, 130a-b is typically generally cylindrical, as shown in FIG. 3B, although the shape (as well as the materials making up the core member and the core member size) may be varied to obtain desired physical or performance properties. For example, the core member 130 shape, size, and materials will directly affect the degree of flexion, extension, lateral bending, and axial rotation of the prosthetic disc. By way of comparison, the dual core structure of FIG. 3B provides a design that includes more space for fibers 140 to be incorporated, thereby providing an additional point of design flexibility.

The annular capsule 150 is preferably made of polyurethane or silicone and may be fabricated by injection molding, two-part component mixing, or dipping the endplate-core-fiber assembly into a polymer solution. As shown, the annular capsule is generally oblong having generally straight sidewalls. Alternative embodiments may include one or more bellows formed in the sidewalls. A function of the annular capsule is to act as a barrier that keeps the disc materials (e.g., fiber strands) within the body of the disc, and that keeps natural in-growth outside the disc.

Several alternative embodiments of the prosthetic discs, and component parts and features thereof, are described and illustrated in FIGS. 4A-E, 5A-B, 6A-C, 7-10, and 11A-D. Turning first to FIGS. 4A-E, the prosthetic disc shown there includes upper and lower endplates 110, 120, each including a pair of inner assemblies 113a-b, 123a-b. Each of the upper endplate 110 and the lower endplate 120 includes a pair of anchoring features 111a-b, 121a-b, respectively. A pair of core members 130a-b are located between the upper and lower endplates 110, 120. Although not shown in the drawings, a plurality of fibers 140 extend between and wrap around the apertures 124 provided on the inner assemblies 113a-b, 123a-b, thereby interconnecting the pair of endplates.

Turning to FIGS. 4B-C, additional detail concerning the construction of the endplates 110, 120 is illustrated. As shown, the inward facing portion of each endplate 110, 120 includes a pair of recesses 115a-b, 125a-b in which the inner assemblies 113a-b, 123a-b are received and attached. Each endplate also includes a central hole 116, 126 through which a portion of each of the inner assemblies 113a-b, 123a-b extends to facilitate connecting the inner assemblies 113a-b, 123a-b to the endplates 110, 120. The inner assemblies 113a-b, 123a-b are preferably attached to the endplates 110, 120 by welding, by use of adhesives, or other suitable method known to those skilled in the art.

FIGS. 4D-E illustrate additional detail concerning the inner assemblies 113, 123. As shown in FIG. 4D, for example, an inner assembly 113 includes a plurality, such as thirteen, apertures 124 around its periphery. The number of apertures generally ranges from about 3 to 100 apertures, preferably in the range of 10 to 30. The apertures 124 may be generally oblong, as shown, or they may be of any other suitable shape or size, as described in other examples herein. FIG. 4E, in addition, illustrates a pair of apertures 124 formed in the central portion of the inner assembly 113. In this embodiment, a fiber 124 may be routed through the center of the core member 130 in addition to the fibers 124 attached to the endplates 110, 120 around the periphery of the core member 130.

Although the inner assemblies 113, 123 shown in the embodiment illustrated in FIGS. 4A-E are generally round, they may also be provided in generally any shape or orientation. The round shape is preferred when it is used in conjunction with a generally cylindrical core member 130, or with a core member 130 otherwise having a generally round footprint. When the inner assemblies 113, 123 are provided in other shapes or sizes, it is preferred to similarly change the shape and/or size of the recesses 115 provided on the inner surfaces of the endplates 110, 120 to accommodate the inner assemblies.

As noted above, although not shown in the drawings, one or more fibers 140 extend between and interconnect the two endplates 110, 120, preferably by being routed through the apertures 124 formed on each of the inner assemblies 113, 123. The fibers 140 may be formed of any of the materials described above, and wound in any suitable pattern described herein or elsewhere to provide desired results. In addition, an optional annular capsule 150 (also not shown in FIGS. 4A-E) may be provided around the perimeter of the space between the two endplates 110, 120, in a manner like that described above in relation to FIGS. 3A-B.

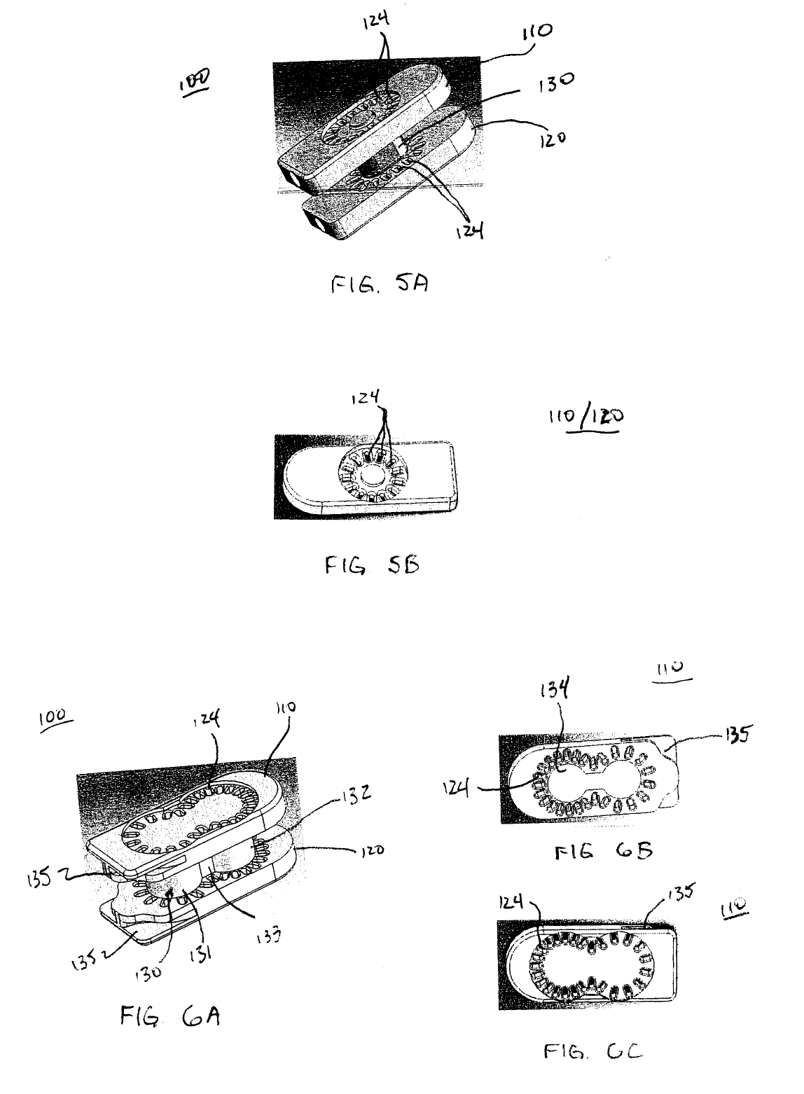

Turning next to FIG. 5A, an alternative embodiment of a prosthetic disc 100 includes endplates 110, 120 having an integrated structure, i.e., without inner assemblies. In the embodiment shown, each endplate 110, 120 is provided with a central portion having apertures 124 forming an oval pattern to accommodate a generally oval, or oblong, shaped core member 130. The apertures 124 may be provided in other shapes and other sizes as well. For example, in FIG. 5B, an integrated endplate 110 is shown having a plurality of apertures 124 forming a generally round pattern, preferably to accommodate a generally cylindrical core member. FIGS. 6A-C, described below, shows a disc 100 having integrated endplates 110, 120 having a plurality of apertures 124 forming a generally barbell-shaped pattern, preferably to incorporate a similarly shaped core member 130. Other shapes and sizes are also possible.

Where the integrated endplates 110, 120 shown in FIGS. 5A-B are used, it is preferred to place a cover or other member (not shown) over the exposed apertures 124 on the upper surface of the upper endplate 110 and over the lower surface of the lower endplate 120. The cover or other member may be formed of the same material as the endplates 110, 120, or it may be formed of a suitable polymeric or other material. Among other functions, the cover would provide protection to the fibers 140 wound around the apertures 124 formed on the integrated endplates. The cover could also have the anchoring features integrated into it.

As shown in FIGS. 5A-B, the lateral, or horizontal, surface area of each of the endplates 110, 120—i.e., the surface area of the surfaces that engage the vertebral bodies—is preferably substantially larger than the cross-sectional surface area of the core member 130. Preferably, the cross-sectional surface area of the core member 130 is from about 5% to about 80% of the cross-sectional area of a given endplate 110, 120, more preferably the range is from about 10% to about 60%, and most preferably from about 15% to about 50%. In this way, for a given core member 130 having sufficient compression, flexion, extension, rotation, and other performance characteristics but having a relatively small cross-sectional size, the core member may be used to support endplates having a relatively larger cross-sectional size in order to help prevent subsidence into the vertebral body surfaces. In the embodiments described herein, the core members 130 and endplates 110, 120 also have a size that is adapted for implantation by way of posterior access or minimally invasive surgical procedures, such as those described above.

Turning next to FIGS. 6A-C, a prosthetic disc 100 having integrated endplates 110, 120 are provided with a core member 130 having a generally barbell shape, including a posterior cylindrical section 131, an anterior cylindrical section 132, and a middle bridging section 133. The inner surface of the upper endplate 110 is shown in FIG. 6B, where it is shown that the endplate 110 is provided with a recess 134 section having a mating keyhole shape for receiving the core member 130. A plurality of apertures 124 are provided on each of the upper endplate 110 and lower endplate 120. The apertures 124 are located on the endplates 110, 120 in a pattern that tracks the periphery of the core member 130. Thus, the fibers 140 (not shown, see FIGS. 3A-B) are routed through the apertures 124 around the core member 130 to interconnect the upper and lower endplates 110, 120. An optional capsule (also not shown, see FIGS. 3A-B) may be provided around the periphery of the fibers 140 and core member 130.

An engagement mechanism 135 is provided at the posterior end of each of the upper and lower endplates 110, 120 of the prosthetic disc. The engagement mechanism 135 provides a surface orientation that allows a tool or other implement to engage the prosthetic disc 100 in order to manipulate the disc during the implantation procedure. For example, the engagement mechanism 135 may comprise a hole, a ledge, a aperture, a tab, or other structure formed on the end of one or both of the endplates 110, 120. In the embodiment shown in FIGS. 6A-C, the engagement mechanism 135 includes a pair of apertures on each of the upper endplate 110 and lower endplate 120. The apertures are adapted to engage tabs formed on a suitable deployment tool.

The apertures 124 formed on the endplates 110, 120 may be provided having any desired density, and the density of apertures may vary over different sections of the endplates 110, 120. For example, the aperture density is higher at the anterior ends of the endplates 110, 120 shown in FIGS. 6A-C than the aperture density of the posterior ends of the endplates 110, 120. For example, fifteen apertures 124 are shown surrounding the anterior portion 132 of the core member 130, whereas only ten apertures surround the posterior portion 131 of the core member 130. In general, the higher fiber density, enabled by a higher aperture density, will provide a higher degree of resistance to flexion, extension, bending and rotation. Aperture densities may be varied in any suitable manner to provide the desirable clinical results.

FIGS. 7-10 illustrate several embodiments of integrated endplates 110 having different shapes, sizes, and orientations. Each of these examples is a portion of a complete prosthetic disc having a similarly sized and shaped lower endplate 120, a core member 130, fibers 140 wound between and interconnecting the endplates, and an optional protective capsule 150, none of which is shown in FIGS. 7-10. Instead, for clarity, FIGS. 7-10 show only the top endplates 110 of the subject prosthetic discs, it being understood that the remaining structure may incorporate any of the features described elsewhere herein.

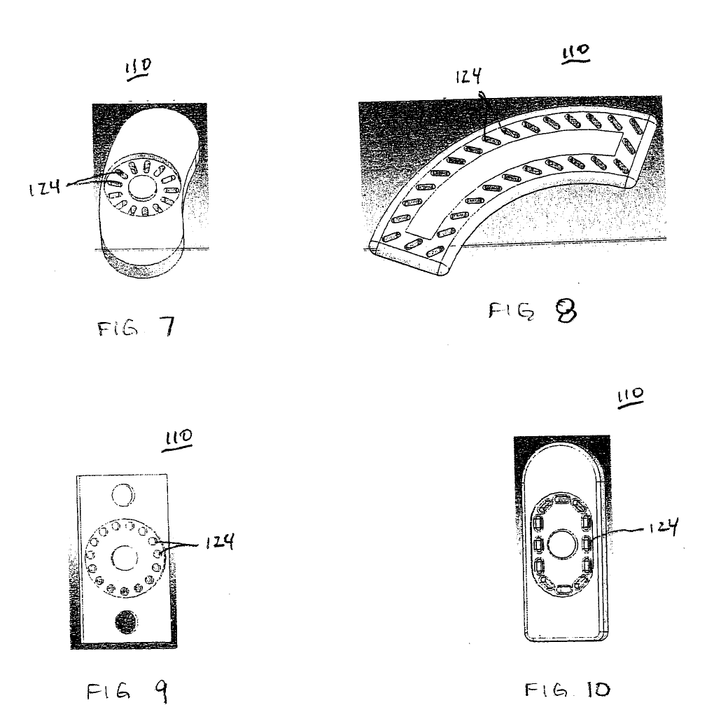

FIG. 7, for example, illustrates a kidney-shaped integrated endplate 110, and FIG. 8 illustrates a curvilinear integrated endplate 110. Each of these shapes includes a curve or curvature that is adapted to approach or approximate the outer curvature of the vertebral bodies and facilitate insertion of the device. Thus, the load borne by the endplates may be distributed outward from the central portion of the vertebral bodies to the shell (ring apophysis) of the vertebral bodies.

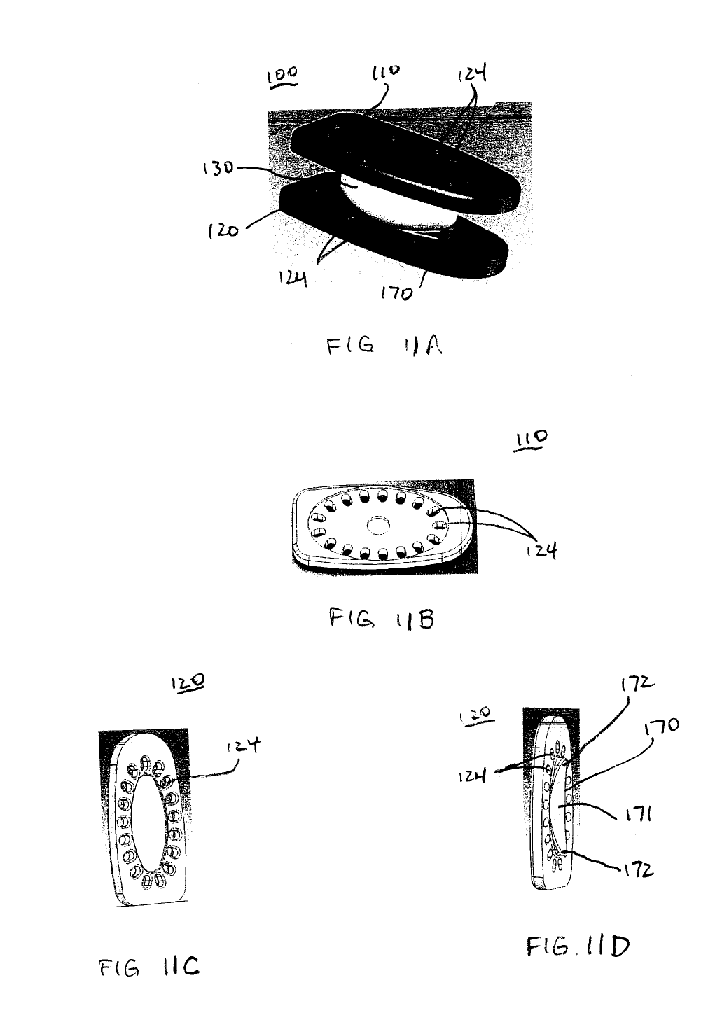

FIG. 9 shows a generally rectangular integrated endplate having round apertures 124. The apertures 124 for winding fibers 124 may be round, as shown in FIG. 9, oblong, as shown in several of the other FIGS., including FIGS. 7, 8, and 10, or of any other suitable shape. The apertures 124 may also be of any size suitable for receiving the fiber 140 windings. FIG. 10, for example, shows a bullet-shaped endplate 110 having a recess adapted to receive a generally oblong-shaped core member 130, and a pattern of generally oblong apertures 124 adapted to surround the periphery of such a core member 130.

The shapes, sizes, and orientations of each of the foregoing endplates are for illustrative purposes only. Additional shapes and sizes are contemplated and are fully in keeping with the prosthetic disc structures described herein.

Turning finally to FIGS. 11A-D, another embodiment of a prosthetic disc 100 is illustrated. The disc includes an upper endplate 110, lower endplate 120, and a core member 130 located between the upper and lower endplates. One or more of the upper endplate 110 and lower endplate 120 includes a curved bearing surface 170. In the illustrated example, only the lower endplate 120 includes a curved bearing surface 170. However, such a bearing surface may be included on the upper endplate 110 instead of, or in addition to, the lower endplate 120. Where only one endplate includes the curved bearing surface 170, the other endplate will preferably be flat. Each of the endplates 110, 120 is generally bullet-shaped, providing for a generally oval shaped core member 130, and a similar oval-shaped pattern for the apertures 124.

The curved bearing surface 170 includes a generally flat middle section 171 and raised sides 172 on either end, approaching the posterior and anterior ends of the endplate 110. The curved bearing surface 170 allows a relative sliding motion between the core 130 and the endplate 120 during flexion and extension of the disc. This also provides for a relatively larger effective core footprint.

It is evident from the above discussion that the present invention provides significantly improved prosthetic intervertebral discs. Significantly, the subject discs closely imitate the mechanical properties of the fully functional natural discs that they are intended to replace.

More specifically, the modes of spinal motion may be characterized as compression, shock absorption (i.e., very rapid-compressive loading and unloading), flexion (forward) and extension (backward), lateral bending (side-to-side), torsion (twisting), and translation and sublaxation (motion of axis). The prosthetic discs described herein are similar to the native physiological constraint for each mode of motion, rather than completely constrain or allow a mode to be unconstrained. In this manner, the present prosthetic discs closely mimic the performance of natural discs.

The subject discs exhibit stiffness in the axial direction, torsional stiffness, bending stiffness in the saggital plane, and bending stiffness in the front plane, where the degree of these features can be controlled independently by adjusting the components of the discs. The interface mechanism between the endplates and the core members of several embodiments of the described prosthetic discs enables a very easy surgical operation. In view of the above and other benefits and features provided by the subject inventions, it is clear that the subject inventions represent a significant contribution to the art.

It is to be understood that the inventions that are the subject of this patent application are not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions. For example, and without limitation, several of the embodiments described herein include descriptions of anchoring features, protective capsules, fiber windings, and protective covers covering exposed fibers for integrated endplates. It is expressly contemplated that these features may be incorporated (or not) in those embodiments in which they are not shown or described.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present inventions, the preferred methods and materials are herein described.

All patents, patent applications, and other publications mentioned herein are hereby incorporated herein by reference in their entireties. The patents, applications, and publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A prosthetic intervertebral disc comprising:
a first endplate;
a second endplate;
a compressible core member positioned between said first and second endplates; and
at least one fiber extending between and engaged with said first and second endplates;
wherein said endplates and said core member are held together by said at least one fiber in a manner which substantially mimics the functional characteristics of a natural intervertebral disc; and
wherein at least one of said first endplate and said second endplate includes a plurality of apertures formed therein at locations substantially displaced from the edges thereof.

2. The prosthetic intervertebral disc of claim 1, wherein both of said first endplate and said second endplate includes a plurality of apertures formed therein at locations substantially displaced from the edges thereof.

3. The prosthetic intervertebral disc of claim 2, wherein said at least one fiber extends through at least one of said apertures of said first endplate and through at least one of said apertures of said second end plate.

4. The prosthetic intervertebral disc of claim 3, wherein said at least one fiber extends through each of said plurality of apertures of said first endplate and through each of said plurality of apertures of said second end plate.

5. The prosthetic intervertebral disc of claim 4, wherein said at least one fiber is wrapped about said endplates thereby defining a unidirectional wrapping pattern.

6. The prosthetic intervertebral disc of claim 4, wherein said at least one fiber is wrapped about said endplates thereby defining a bidirectional wrapping pattern.

7. The prosthetic intervertebral disc of claim 4, wherein said at least one fiber is wrapped about said endplates thereby defining a multi-directional wrapping pattern.

8. The prosthetic intervertebral disc of claim 4, wherein said at least one fiber defines two or more layers of fibers.

9. The prosthetic intervertebral disc of claim 8, wherein the fibers of a first layer and the fibers of a second layer are applied with the same tension.

10. The prosthetic intervertebral disc of claim 8, wherein the fibers of a first layer and the fibers of a second layer are applied with different tensions.

11. The prosthetic intervertebral disc of claim 8, wherein said fibers of a first layer extend at a first angle relative to at least one of said endplates, and said fibers of a second layer extend at a second angle relative to the same at least one of said endplates, wherein said first angle is different than said second angle, and further wherein said angles are selected to mimic the fibers of a natural disc.

12. The prosthetic intervertebral disc of claim 1, wherein said compressible core member comprises one of either polyurethane or silicone.

13. The prosthetic intervertebral disc of claim 1, wherein said at least one fiber comprises an elastomer.

14. The prosthetic intervertebral disc of claim 1, wherein said at least one fiber comprises a metal.

15. The prosthetic intervertebral disc of claim 1, wherein said at least one fiber comprises a plastic.

16. The prosthetic intervertebral disc of claim 1, wherein said at least one fiber is a multifilament fiber.

17. The prosthetic intervertebral disc of claim 1, wherein said at least one fiber is a monofilament fiber.

18. The prosthetic intervertebral disc of claim 1, wherein said at least one fiber is encapsulated.

19. The prosthetic intervertebral disc of claim 1, further comprising a fixation member for securing said first endplate to a vertebral body, said fixation member extending from an outer surface of said first endplate.

20. The prosthetic intervertebral disc of claim 19, wherein said fixation member comprises at least one anchoring feature.

21. The prosthetic intervertebral disc of claim 1, further comprising a capsule encasing said compressible core.

22. The prosthetic intervertebral disc of claim 21, wherein said capsule is bellowed.

23. The prosthetic intervertebral disc of claim 1, wherein at least one of said first endplate and said second endplate includes a curved bearing surface engaged with said core member.

24. A prosthetic intervertebral disc comprising:
a first endplate;
a second endplate;
a compressible core member positioned between said first and second endplates; and
at least one fiber extending between and engaged with said first and second endplates;
wherein said endplates and said core member are held together in a manner which substantially mimics the functional characteristics of a natural intervertebral disc; and
wherein at least one of said first endplate and said second endplate includes a curved bearing surface engaged with said core member.

25. The prosthetic intervertebral disc of claim 24, wherein both of said first endplate and said second endplate includes a curved bearing surface.

26. The prosthetic intervertebral disc of claim 24, wherein said curved bearing surface comprises a generally flat middle section and a raised side on each opposed end of said at least one of said first endplate and said second endplate.
